(19) United States
(12) Patent Application Publication
(10) Pub. No.: US 2022/0208385 A1
(43) Pub. Date: Jun. 30, 2022

(54) PERSONALIZED MESSAGING SYSTEM FOR INCREASING SUBJECT ADHERENCE OF CARE PROGRAMME

(71) Applicant: Well-Beat Ltd., Kfar-Saba (IL)

(72) Inventors: David VOSCHINA, Kfar-Saba (IL); Jackie ASSA, Tel Aviv (IL)

(73) Assignee: Well-Beat Ltd., Kfar-Saba (IL)

(21) Appl. No.: 17/564,379

(22) Filed: Dec. 29, 2021

Related U.S. Application Data
(60) Provisional application No. 63/131,337, filed on Dec. 29, 2020.

(51) Int. Cl. G16H 50/30

(52) U.S. Cl. CPC G16H 50/30; G16H 10/20

(57) ABSTRACT

The disclosure is of a system and a method for increasing the duration and quality of adherence in subjects by automatically managing creation and delivery of personalized, psychological and motivational profile state focused, messages.

The disclosure comprises a recommendation engine, supporting several states, based on data from data collectors such as questionnaires and data collected form handled and wearable devices, and knowledge representation of expert guidelines.

Thereby personalized interventions using digital messages, involving other people such as family or medical staff, using multimedia, and/or the like may be applied.

### RELATED APPLICATION(S)

This application claims the benefit of priority under 35 USC § 119(e) of U.S. Provisional Patent Application No. 63/131,337 filed on Dec. 29, 2020, the contents of which are incorporated by reference as if fully set forth herein in their entirety.

### FIELD AND BACKGROUND OF THE INVENTION

The present invention, in some embodiments thereof, relates to generating messages for user interaction, and, more particularly, but not exclusively, to generating messages for adherence and motivational increase of care program subjects.

Medical and well-being procedures may require subject to perform activities following a strict care program. Examples of such procedures include, for example, protocols for rehabilitations of stroke subjects, diet supporting routines for weight watchers, and sport and activities regime for people with obesity risk.

Although such policies are designed to increase the welfare of the subject over time, low adherence levels of the subjects is common, both in the adherence compliance and in its actual period length. This behavior introduces sub-optimal success of the policies, for example recovery failures and re-hospitalization in cases of rehabilitation, or weight gain in cases of failed diet plans.

A major method for improving subject's adherence and motivation for following the policy guidelines, may comprise continuously giving the subject motivational and encouraging messages and notifications. It was shown that such messages have significant effect on the subject motivation, and as a result have significant on the adherence time and quality. Such messages should be managed carefully, as too many messages, or messages that are misaligned with the subject psychological state, may cause actual deterioration in the adherence.

One way to determine the effective messages quantity and quality for a specific subject psychological state, is by copying similar adherence increasing messages for similar subject profiles, and avoiding adherence reducing messages of subjects with similar profiles. Similar method in e-commerce is called user-based recommendation services, which utilize purchase recommendation results for users to increase purchase effectiveness of similar future users.

However, determining the subject psychological and motivational state, realizing the effectiveness of messages, and inferring the influence between the two towards adherence improvement, pose challenges that are difficult to handle with limited per-person time caregiver personnel especially for large groups of subjects.

### SUMMARY OF THE INVENTION

It is an object of this disclosure to provide a system and a method for personalizing messages, using a collaborative filtering mechanism based recommender and an expert-in-the-middle.

According to an aspect of some embodiments of the present invention there is provided a system for generating motivational interactions for increasing a subject adherence to a prescribed treatments, comprising at least one hardware processor configured to:

extract a plurality of metrics estimating the subject psychological and motivational traits using data collectors;

use a profiler to extract a subject profile, based on the plurality of metrics and a plurality of internally stored details;

use a collaborative filtering mechanism based recommender to generate a plurality of recommended personalized interventions;

apply a plurality of rules using an expert-in-the-middle on the plurality of recommended personalized interventions; and

generate motivational interactions according to the plurality of recommended personalized interventions.

According to an aspect of some embodiments of the present invention there is provided a method of generating motivational interactions for increasing a subject adherence to a prescribed treatments, the method comprising:

extracting a plurality of metrics estimating the subject psychological and motivational traits using data collectors;

using a profiler to extract a subject profile, based on the plurality of metrics and a plurality of internally stored details;

using a collaborative filtering mechanism based recommender to generate a plurality of recommended personalized interventions;

applying a plurality of rules using an expert-in-the-middle on the plurality of recommended personalized interventions; and

generating motivational interactions according to the plurality of recommended personalized interventions.

Optionally, the extracting a plurality of metrics comprises analyzing specialized self-questionnaire.

Optionally, further comprising data collectors, comprising tracing the subject acknowledgement of the motivational interactions.

Optionally, the internally stored details comprise a weighted analysis of previous subject's adherence to the prescribed treatments, using the data collectors.

Optionally, further comprising initializing an inference of effectiveness of the plurality of recommended personalized interventions using expert-in-the-middle.

Optionally, the motivational interactions comprise motivational and informational messages and notifications to the subject.

Optionally, further comprising initializing the collaborative filtering mechanism based recommender using the expert-in-the-middle.

Optionally, further comprising adjusting the collaborative filtering mechanism based recommender using at least one questionnaire answered by the subject.

Optionally, further comprising initializing the collaborative filtering mechanism based recommender using specialized cold start mechanism.

Optionally, generating motivational interactions or extracting a plurality of metrics further comprising using a chat-bot configured by the subject profile.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings and formulae. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of an exemplary system generating messages for user interaction, according to some embodiments of the present disclosure;

FIG. 2 is a flowchart of an exemplary process for generating messages for user interaction, according to some embodiments of the present disclosure;

FIG. 3 is a schematic diagram of an exemplary system for generating messages for user interaction, according to some embodiments of the present disclosure;

FIG. 4A is a basic flow chart of an additional exemplary process generating messages for user interaction, according to some embodiments of the present disclosure;

FIG. 4B is a basic flow chart of an exemplary recommender system state transition in a system for generating messages for user interaction, according to some embodiments of the present disclosure;

FIG. 5 is a basic flow chart of a further additional exemplary process generating messages for user interaction, according to some embodiments of the present disclosure;

FIG. 6 is an exemplary chart of exemplary subject traits, according to some embodiments of the present disclosure; and

FIG. 7 is a basic flow chart of an exemplary month of an exemplary care program, according to some embodiments of the present disclosure.

### DESCRIPTION OF SPECIFIC EMBODIMENTS OF THE INVENTION

The present invention, in some embodiments thereof, relates to generating messages for user interaction, and, more particularly, but not exclusively, to generating messages for adherence and motivational increase of care program subjects.

The disclosure includes a system and a method for increasing the duration and quality of adherence in subjects by automatically managing creation and delivery of personalized, psychological and motivational profile state focused, messages.

Some embodiments of the present disclosure comprise data collectors such as questionnaires, data collected form handled and wearable devices, subject history, and electronic medical records (EMR) interaction summaries.

Some embodiments of the present disclosure comprise a recommendation engine, supporting several states, based on the quantity and quality of data collected from the subject, and knowledge representation of expert guidelines. The recommendation engine may characterize several traits of the subject and use the engine together with an expert-in-the-middle to determine personalized interventions.

Some embodiments of the present disclosure apply the personalized interventions using digital messages, involving other people such as family or medical staff, use multimedia, operate external devices.

For better understanding of the detailed description, a set of terms is defined for describing some of their details of the examples, however additional examples may be developed in the future or may benefit from the disclosed method and the examples provided for the terms should not to be construed as limiting the scope of the claims.

As used herein, the term care program refers to the method to which adherence is to be improved such as health care program. This may include but not limited to, medical and health related procedures, such as medication consumption, rehabilitation protocols, exercise and diet regime, changing habits, handling infection preventive quarantine routines and more. The policy usually includes routine activities that should be followed by the subject over long periods of time. The method may also be used for competitive sport training, learning, and the like.

As used herein, the term adherence duration and quality refers the metrics in which the adherence for the required care program are measured. Subjects usually tend to reduce their compliance to a specific care program over time, either by partially performing the required policy, or by completely disregarding the required policy. The duration and quality of following the rules over time, is being used to measure the effectiveness of the increasing adherence method.

As used herein, the term subject profile refers to traits such as the subject psychological and motivational traits that are estimated and used by the system. This includes but is not limited to several known properties in the psychology literature, such as readiness, persistence, self-efficacy, approach, motivation, and barrier.

As used herein, the term caregiver personnel refers to the professional team assisting the subject to comply with the care program. The team structure and responsibilities vary according to the different domain and policies. For example, for diet control and maintenance, the caregiver personnel are the prescribing dietitians and their team. In rehabilitation scenarios the caregiver personnel include the physicians the nurses, the caregiver and medical call-center personnel and more.

As used herein, the term messages refers to the delivery of digital messages to the subject mobile device, personal computer, personal assistant, and the like. The message types may include but is not limited to, informative information regarding the care program, motivational tips, references to academic and public articles related to the treatment, and details about achievements of the subject in performing activities related to the care program. Messages may be sent as stand-alone messages or integrated into other applications, for example chats conducted by a chat-bot.

As used herein, the term dialogue scripts refers to tools of a sales representative, including sentences, phrases, and arguments that should be used by the agent, to engage the customer, by replicating the success of previous sales. Here, we extend this term to care personnel, where the dialogues should be geared to extend and improve adherence. This includes guiding the agent with specific sentences, part of sentences, arguments that should be used, and arguments that should not be used, conversation tone and more.

As used herein, the term recommendation engine refers to a software component that is used in businesses to select from a given set of items, and subjects, the best item for a certain subject, to maximize a business goal. For example, recommending the best suitable movie for a person, or recommending most likely to buy items, for a purchase by an individual. The recommendation engine may be applied for offering messages, and specific dialogue scripts that would generate the biggest impact in increasing compliance to a care program.

As used herein, the term chat-bot mechanisms refers to methods to directly collect data from the subject, for example about regular consumption of prescription drugs, diet, and physical activity. The chat-bots may be built on existing application and comprise several questions which may be selected from large list of topics accordingly to their importance level, enabling free text messages, or multiple choice questions. The chat-bots may also use voice call based on the combination of the text bots with text-to-voice tool.

As used herein, the term adherence score refers to weighting techniques enabling, based on the importance level attributed per each data type, to combine all the raw data into few numbers, or a single number per a given number of days.

Before explaining at least one embodiment of the invention in detail, it is to be understood that the invention is not necessarily limited in its application to the details of instructions and the arrangement of the components and/or methods set forth in the following description and/or illustrated in the drawings and/or the Examples. The invention is capable of other embodiments or of being practiced or carried out in various ways.

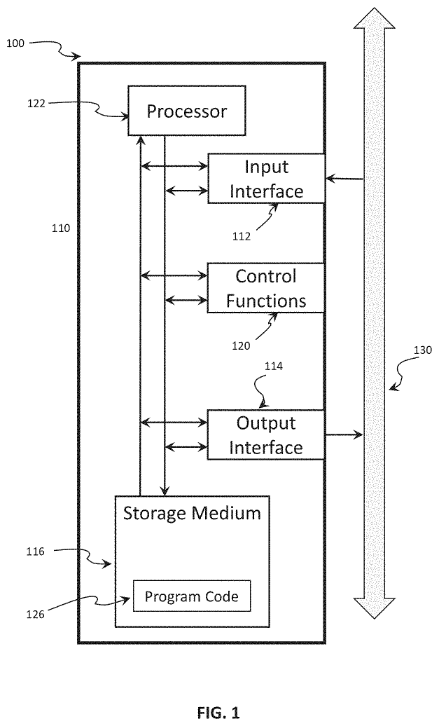

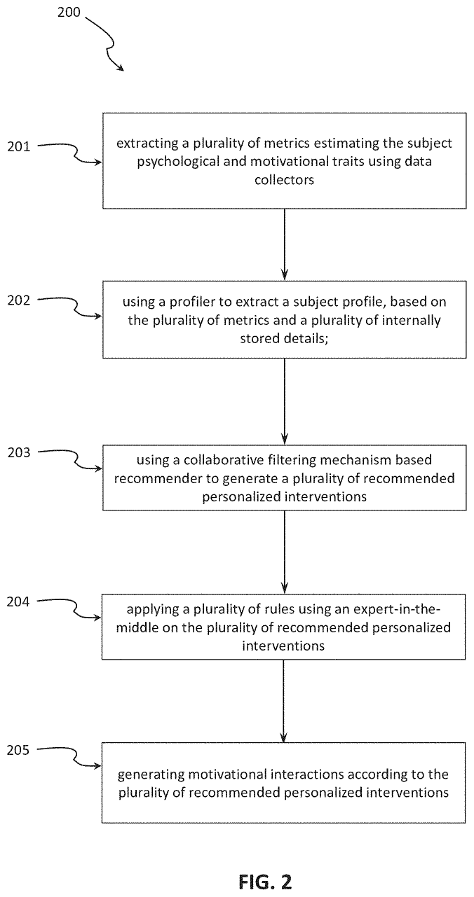

Referring now to the drawings, FIG. 1 is a schematic illustration of an exemplary system generating messages for user interaction, according to some embodiments of the present invention. An exemplary messages generating system 100 may execute processes such as 200 for generating messages for user interaction. Further details about these exemplary processes follow as FIG. 2 is described.

The messages generating system 110 may include a set of interfaces to a network, as well as other devices and instruments. The interfaces may comprises an input interface 112, and an output interface 114. The messages generating system may also comprise one or more processors 122 for executing processes such as 200, and storage 116, comprising a portion for storing code (program code storage 126) and/or memory for data, such as device and/or machine parameters, control scenarios, and/or the like. The messages generating system may be physically located at the subject's home, implemented on a mobile device, implemented as distributed system, implemented virtually on a cloud service, on machines also used for other functions, and/or by several options. Alternatively, the system, or parts thereof, may be implemented on dedicated hardware, FPGA and/or the likes. Further alternatively, the system, or parts thereof, may be implemented on a server, a computer farm, the cloud, and/or the likes. For example, the storage 116 may comprise a local cache on the device, and some of the less frequently used data and code parts may be stored remotely.

The input interface 112, and the output interface 114 may comprise one or more wired and/or wireless network interfaces for connecting to one or more networks, for example, a local area network (LAN), a wide area network (WAN), a metropolitan area network (MAN), a cellular network, the internet, a combination thereof, and/or the like. The input interface 112, and the output interface 114 may further include one or more buses 130. The buses may comprise wired and/or wireless interconnection interfaces, for example, a universal serial bus (USB) interface, a serial port, and/or the like. Furthermore, the output interface 114 may include one or more wireless interfaces for generating messages using varied media, and the input interface 112, may include one or more wireless interfaces for receiving information from one or more devices. Additionally, the input interface 112 may include specific means for communication with one or more sensor devices such as a camera, microphone, motion sensor, medical sensor sensing indicators such as pulse, temperature, oxygen saturation, other biometric sensors, weather sensor and/or the like. And similarly, the output interface 114 may include specific means for communication with one or more display devices such as a loudspeaker, display and/or the like.

The one or more processors 122, homogenous or heterogeneous, may include one or more processing nodes arranged for parallel processing, as clusters and/or as one or more multi core one or more processors. The storage 116 may include one or more non-transitory persistent storage devices, for example, a hard drive, a Flash array and/or the like. The storage 116 may also include one or more volatile memory devices, for example, a random access memory (RAM) component, enhanced bandwidth memory such as video RAM (VRAM), and/or the like. The storage 116 may further include one or more network storage resources, for example, a storage server, a network attached storage (NAS), a network drive, and/or the like accessible via one or more networks through the input interface 112, and the output interface 114.

The one or more processors 122 may execute one or more software modules such as, for example, a process, a script, an application, an agent, a utility, a tool, an operating system (OS) and/or the like. The software modules may comprise a plurality of program instructions stored in a non-transitory medium within the program code 126, which may reside on the storage medium 116. For example, the one or more processors 122 may execute a process, comprising generating messages such as 200 and/or the like. This processor may generate text messages, voice messages, multimedia messages, display graphics, cause a device to vibrate or release an odor, and interact with one or more subject, or people designated to help facilitate the care program, using one, some, or all the of senses.

Referring now to FIG. 2, which is a flowchart of an exemplary process for generating messages for user interaction, according to some embodiments of the present disclosure. The processor 122 may execute the exemplary process 200 for a variety of purposes involving generating messages for user interaction for medical purpose, marketing, training and/or the like.

Alternatively, the process 200 or parts thereof may be executing using a remote system, an auxiliary system, and/or the like.

The exemplary process 200 starts, as shown in 201, with extracting a plurality of metrics estimating the subject psychological and motivational traits using data collectors.

The metrics may be personal traits, characteristics, which may be named such as readiness, self-efficacy, persistence, sociability, and the like, as well as embeddings and traits not easily convertible to natural language. The data collectors, may comprise tracing the subject data from the subject wearable device or mobile phone, digital questionnaires, digital surveys, and the like. When the subject is a medical patient, and/or in some cases of competitive sport training, medical records of the subject, such as former diseases, blood test results, pulse measurements, oxygen saturation, heartbeat, and the like may also be measured. For a marketing focus group, traits such as hobbies and former purchases may be used by the data collectors.

The exemplary process 200 continues, as shown in 202, with using a profiler to extract a subject profile, based on the plurality of metrics and a plurality of internally stored details.

The profile extracted may comprise a plurality of metrics comprises analyzing specialized self-questionnaire, and other data available from the data collectors such as data from the subject wearable device or mobile phone. Medical information, diet, and previous purchases.

When such information is available, the profiler may also apply internally stored details comprising analysis of previous subject's adherence to the prescribed treatments, which may be collected using the data collectors.

The exemplary process 200 continues, as shown in 203, with using a collaborative filtering mechanism based recommender to generate a plurality of recommended personalized interventions.

The collaborative filtering mechanism may map subjects to a hyperspace characterized by their traits, and/or embeddings thereof, and apply proximity measures and/or clustering to infer from which other subjects, assumptions may be made about other traits of the subject, predicted responses to interactive messages and compliance with a care program.

Cold start is a known problem of collaborative filtering mechanism based recommender engines, and is characterized by high uncertainty of subject traits, and may also apply to relations between the traits and the effect of interaction options on the adherence pattern to care programs.

Some embodiments may apply Bayesian methods to represent the cold start effect and mitigate it. Some embodiments may comprise initializing the collaborative filtering mechanism based recommender using specialized cold start mechanism. The specialized cold start mechanism may comprise adjusting the collaborative filtering mechanism based recommender using at least one questionnaire answered by the subject, and/or data such as electronic medical records (EMR) and the like. The specialized cold start mechanism may further comprise initializing the collaborative filtering mechanism based recommender using the expert-in-the-middle, which may comprise knowledge representation of cognitive and behavioral psychology, marketing methods, sport training methods, and/or the like by coded rules, a machine learning model trained thereby, and/or the like.

The plurality of recommended personalized interventions may comprise a set of interventions such as direct messages, text messages, multimedia messages, and/or the like, which may be delivered through computerized means such as a home computer, internet of things (IoT) device or a mobile phone, as well as scripts for care staff. Some embodiments may integrate messages in gamification schemes.

The exemplary process 200 continues, as shown in 204, with applying a plurality of rules using an expert-in-the-middle on the plurality of recommended personalized interventions.

The plurality of rules coded in the expert-in-the-middle may be based on care personnel experience, research results, academic literature such as psychology, and/or the like. These rules, for example, may state that an analytic minded subject may respond better to scientific proven advice, rather than emotional motivational statements.

These rules may be programmed directly, or indirectly using a trained machine learning model.

Particularly when the recommender engine is affected by the cold start problem, inference of effectiveness of the plurality of recommended personalized interventions may use the expert-in-the-middle rather than the collaborative filtering mechanism based recommender, or a combination thereof.

And subsequently, as shown in 205, the process 200 may continue by using the system for generating motivational interactions according to the plurality of recommended personalized interventions

Information from the expert-in-the-middle and the recommender engine, may be used to choose motivational interactions such as messages, messaging family members or friends to show involvement, and other intervention methods. The motivational interactions may comprise motivational and informational messages and notifications to increasing compliance to a prescribed treatments for the subject, and may be performed automatically, assisted by care staff, or as a combination thereof.

Additionally, care staff may be notified to get involved through computers or mobile devices such as cellular phones.

It should be emphasized that variants of the process are apparent to the person skilled in the art and are within the scope of the claims.

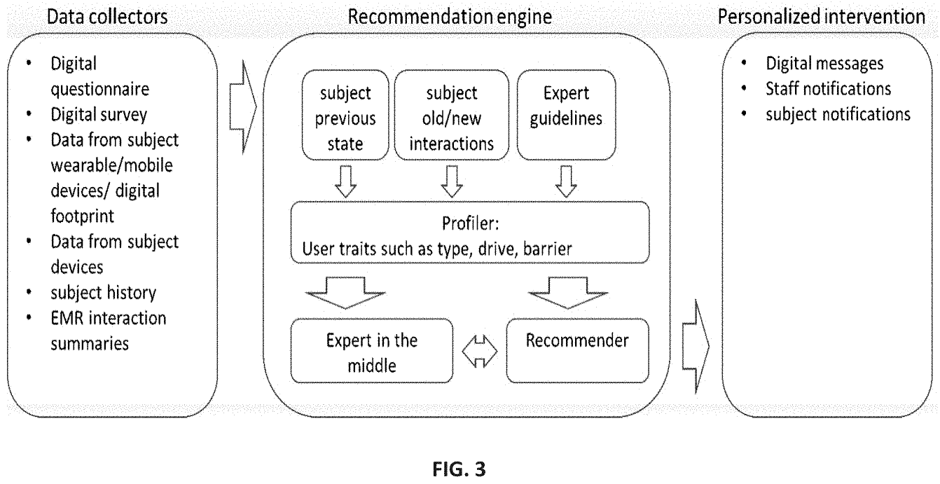

Reference is also made to FIG. 3, which is a schematic diagram of an exemplary system for generating messages for user interaction, according to some embodiments of the present invention.

The diagram introduces a system and a method for increasing the adherence duration and quality of subject, by determining the psychological and motivational state of the subject, and sending the subject messages which were shown to be effective by past subjects with similar states, to extend their adherence duration and quality.

The disclosure enables personally adapted messaging as part of intervention, using a recommendation engine, which maximizes effectiveness by repeating such inputs which had positive adherence results on subjects with similar psychological and motivational profiles.

The disclosure comprises the collection of data and feedback from the subject, and the care staff, the recommendation of the personalized intervention, and the creation, delivery, notification, and management of these inputs. During initial stages, when the result of only a few recommendations is available, also referred to as ‘Cold start’ state, the system may use expert defined set of rules to determine the used recommendations. Following some usage, the data on the interactions and messages based on the system recommendations may become statistically significant, which enables the system to automatically update the recommendations adherence success statistics according to the associated message or interaction type success after profile updates.

The system may comprise three major components, ‘Data Collectors’, ‘Recommendation engine’, and ‘Personalized intervention’.

The Data Collectors may collect and control the information used as input to the system. For example includes the information may comprise one or more digital questionnaires: During the joining process of the care group, each subject may fill in an initial questionnaire. The questionnaire includes a short list of questions designed to capture the subject preferences and opinions and be used to infer, for example by the system recommendation profiler, the subject current psychological and motivational state.

The Data Collectors may further collect subject data: The system may collect data from the subject wearable device, mobile phone and other devices, to infer relevant information related to the psychological and motivational state. This may include, for example, the user location, activities, group activities and more. Additional data may include digital feedback questions related to satisfaction of previous interventions that the subject may submit.

The Data Collectors may further conduct one or more digital Surveys: In order to verify and substantiate the determined subject profile, the system may request one or more subjects to submit a digital survey about their preferences and current state. The result of this survey may be analyzed by the recommendation engine profiler to validate the current subject profile state.

The Data Collectors may further collect subjects' history: subject interaction history with the system, with the care staff, and relevant medical and social history create richer psychological and motivational profile of the subject and enable a better recommendation of the interventions.

The Data Collectors may further apply one or more chat-bot mechanisms to directly collect data from the patient. The data collected may comprise use of medications, diet, training, purchasing products, and/or the like.

The chat-bot which may also be used for generating motivational interactions may be used extracting a plurality of metrics configured by the subject profile

The chat-bot may be stand alone, or based on existing application, and initiate a session comprising several questions, which may be selected according to their importance level, uncertainty, and/or the like. The subject, for example the patient may use free text provide feedback. The chat session may also comprise motivational message based on the subject traits, and current dynamic motivational profile, which may be based on an analytic engine output.

The chat-bot may also comprise using multiple choice questions, wherein the subject may select a feedback from a closed list. The chat-bot may comprise one or more landing pages to apply the multiple choice questions.

The chat-bot may also comprise using voice calls which may apply a text-to-voice tool. Some embodiments may use augmented reality, virtual reality, and/or the like, to use an avatar for enhancing the feel of the chat-bot.

The chat-bot may be configured by the subject profile. The chat-bot may collect data based on the subject traits or profile, as well as provide motivational messages adapted to the subject's characteristics represented in the subject's profile. The trigger of a chat-bot session may be based on dynamic patient motivational profile, clinical status, levels of certainty about the subject's profile, and/or the like. For example, the frequency may be changed based on the subject's persona type and in order to increase the engagement level by digital intervention thereby personalizing the chat-bot based on the dynamic subject's profile.

The recommendation engine may comprise a profiler, an expert-in-the-middle module, and a recommender module. The recommendation engine may determine the most effective personalized intervention for a specific user, by applying the subject current profile, and considering the effectiveness of the possible interventions of similar profile users.

The recommendation engine profile may apply the collected data from the data collectors, together with internally stored details such as the subject previous state, the subject interactions, and expert guidelines, to infer the subject current psychological and motivational profile. The internally stored details may comprise the subject relevant traits, such as subject type, the subject's drive or motivation, current barrier, as well as trait embeddings which may be difficult to verbalize.

The subject profile may be used to infer the recommended intervention, by the recommender. The determined profiles of subjects may also be used for creating cohorts of similar profile subjects, for example by clustering. The internally stored details of other subjects from the subject's associated cohort may be used to infer recommended interventions for subjects who do not have yet statistically significant intervention recommendations.

The expert-in-the-middle may enable professional experts to define system rules, providing knowledge representation of permitted and non-permitted intervention and intervention details for specific group profiles. These rules may be used for inferring recommendations for these cohorts, and the cohorts' subject's feedback provides the data to determine the recommendation effectiveness and thus control future recommendations.

The expert-in-the-middle may further enable domain experts to examine the statistics of specific recommendations for a specific subject profile, and override the system preference directly, when required.

The recommender module may be based on collaborative filtering mechanism, with specialized adaptation to enable exploration in the initial phase, also referred to as ‘Cold start’, using expert specific rules. The recommender may infer for each subject, the next personalized interventions together with their traits and properties. The collaborative filtering mechanism may apply similarity of recommendations and similarity of subject profiles, to infer success of a specific subject, using past results on subjects having similar profile and traits.

The recommendations may be sent to a personalized intervention module, which manages the delivery and control of the suggested interventions.

The personalized intervention module, may control the intervention delivery, comprising the following examples.

Message notifications are an example of interventions: The message notifications may include several ways to notify the subject upon arrival of a message. For example, changing the lighting conditions in the subject room, having the mobile device read aloud the notification details or a predefined notification message.

The subject acknowledgement of the notification may trigger a digital message rendering, and a feedback data that is sent to the data collector modules. The delivery of messages also notifies the care staff, who may examine the subject past notifications and messages, together with their related feedbacks. The staff notification may be done by the staff display, or using any other device such as a computer, a mobile device, a pager, and/or the like which may generate audio/visual cue or read-aloud the notification details.

Digital messages may comprise the selected motivational messages, for example using different tone, and addressing different barriers and different types of topics for each subject. Messages may be delivered to the subject mobile device, personal computer, wearable device, personal assistance device or other device according to the user preference. These devices may vibrate, display and/or read aloud the messages, upon the subject consent to the notification.

Following user activities and additional device usage data such as the one described in the data collector module, may be used to infer the effectiveness of the message, for example, if the message asks the subject to visit the clinic at a certain date, the actual subject visit to the clinic may confirm the message effectiveness and the subject adherence state.

Note that this schematic diagram is exemplary, and variants apparent to the person skilled in the art are within the scope of the claims,

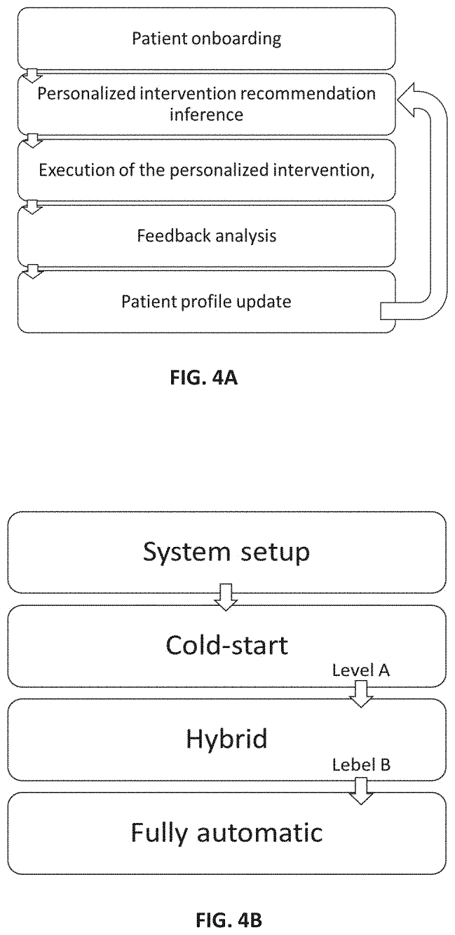

Reference is also made to FIG. 4A which is a basic flow chart of an additional exemplary process generating messages for user interaction, according to some embodiments of the present invention.

The additional exemplary process may be executed for generating messages for increased motivation and adherence by using interaction, for example text messages, multimedia messages, operating variety of devices, and/or the like. The process may be executed by the one or more processors 122, using a central system. A cloud service, and/or the like.

FIG. 4A shows an example of the ongoing set of steps done by the different parts of the system related to each subject to increase his adherence.

The first step of the process is Patient onboarding, or more generally subject onboarding, which comprises receiving and analyzing a digital entry questionnaire that the subject fills. This questionnaire may include a set of questions regarding the subject preferences and current state. The analysis enables creating an initial subject profile, which includes estimation for several subject traits. For examples traits may include: Readiness—how ready the subject is to begin to comply with the care program; Persistence—what is the subject persistence level in performing tasks; Self-efficacy—the subject view on how well one may perform the required activities; Approach—what is the subject approach to fulfilling required activities; Motivation—how motivated is the subject to comply with the activities of the care program; Barrier—what does the subject main barrier to compliance.

These exemplary traits, as well as other traits and embeddings of subject data, may be used for classification of the subject to a set of given known cohorts or profile types such as “adhering to authority”, “adhering to social pressure”, “adhering to logical explanations” and the like.

The Personalized intervention recommendation inference may apply the subject profile, and effectiveness of previous interventions to select the most effective and suitable intervention. The selection may be performed by the recommender part, according the predefined rules of the expert-in-the-middle, and statistics of past interventions of subjects with similar profiles.

The Execution of specific personalized intervention may apply new or updated motivational interactions for a given subject, or follow deterioration in a subject motivational profile, by generating notifications for an assigned care staff member. Following the notification, the care member may initiate a conversation, either face to face or virtual by using phone, chat, or the like, with the subject, in which the member may follow the suggested personalized dialogue script guidelines. In conclusion of the conversation, the care member may generate a report or write a feedback summary of the conversation which is fed back to the system.

The Feedback analysis may apply the returned intervention feedback, together with additional data collected form the subject and the care environment. For example, activities of the user, and clinic visits as noted by the staff personnel, may be collected and analyzed to determine the potential effect on the subject profile.

The Patient profile update, or more generally subject profile update, for example for professional sport trainees or marketing focus group members, may comprise analyzing the feedback together with the subject's, and similar subjects whose profile is of the same cohort. Activities may be fed to generate an updated subject profile, with updated traits properties, after which the recommender may infer the required updates in the ‘Personalized intervention recommendation inference’, for following personalized intervention for the subject.

Reference is also made to FIG. 4B which is a basic flow chart of an exemplary recommender system state transition in a system for generating messages for user interaction, according to some embodiments of the present invention.

FIG. 4B describes an example of the different phases the system advancement for new adherence increasing care service and a new set of subjects. These phases may be required to gain statistical significance over the personalized intervention recommendations, which is initially bootstrapped using the expert-in-the-middle.

More specifically, the system may begin with a System setup phase, in which the messages for potential profiles and subject traits may be defined. During this phase, the integration to the various applicable data collectors may be implemented and tested, optionally comprising a specific questionnaire, surveys, the subject feedback data, and other systems from which the subject history and EMR should be extracted.

In the next, Cold-start phase, an initial set of subjects may join the service. During this phase, the system may analyze the generated profiles, according to the description in the above sections, and apply clustering methods such as Density-Based Spatial Clustering of Applications with Noise (DBSCAN), Hierarchical Density-Based Spatial Clustering of Applications with Noise (HDBSCAN), K-means clustering, grid based clustering, fuzzy clustering, and/or the like to create large clusters of such profiles, which may be delivered to the domain expert for evaluation.

An expert may examine the common profile characteristics and suggest recommendation rules to determine options of the personalized interventions preferred for each profile cluster, according to the expert's professional experience and/or reference literature. This may include both selecting a set of allowed options, and options which are disallowed and may cause negative effect.

For example, the expert may decide that for an “authority uneasy” profile the intervention should not be using a demanding tone, as it may cause deterioration in the motivation of such subjects. These rules may be used during this phase to generate interventions for subjects, by selecting randomly one of the allowed options for each subject.

During this phase, the feedback information about the subject adherence may be collected.

The system may progress to the next, hybrid phase, when the statistical significance of the subject feedback replies reaches a level A threshold (for example when 20% of the interventions have >50% statistically significant success/failure results and the number of allowed alternatives over all rules, had reached another level A threshold (for example 50%). Different thresholds, such as when 40% of the interventions have >50% statistically significant success/failure results, 10% of the interventions have >70% statistically significant success/failure results, may also be used.

During the hybrid phase, the system recommendations may be inferred by considering both the existing expert rules, and the statistically significant intervention. This enables to continue automatically increasing the number of statistically significant interventions while still using expert rules for many subjects for whom no stable or reliable results were measures for similar subject interventions.

Periodically, during this stage, the system may generate sets of subjects of similar profiles, for example assigned to the same cluster, or who share common properties. The domain expert may define new rules for allowed and disallowed interventions to these groups and enrich the options available for the system to provide messages for such subjects.

The system may progress to the next, Fully automatic, phase when the statistical significance of the subject feedback replies reaches level B threshold (for example 50% of the interventions have >50% statistically significant success/failure results and the number of allowed alternatives over all rules, had reached level B threshold (for example 80%). Different thresholds, such as the number of allowed alternatives over all rules, had reached 60% or 90% may be used.

In this stage recommendations may be applied in messages, automatic interventions, and/or notifications to care staff, may be performed completely automatically and additional expert driven rules may not be needed. In the phase, the expert may control the system recommendation by defining overriding rules, which would override the system selection for certain subject profiles.

It should be noted the flow is shown as an example, and different phase partitions may be used and be within the scope the claims.

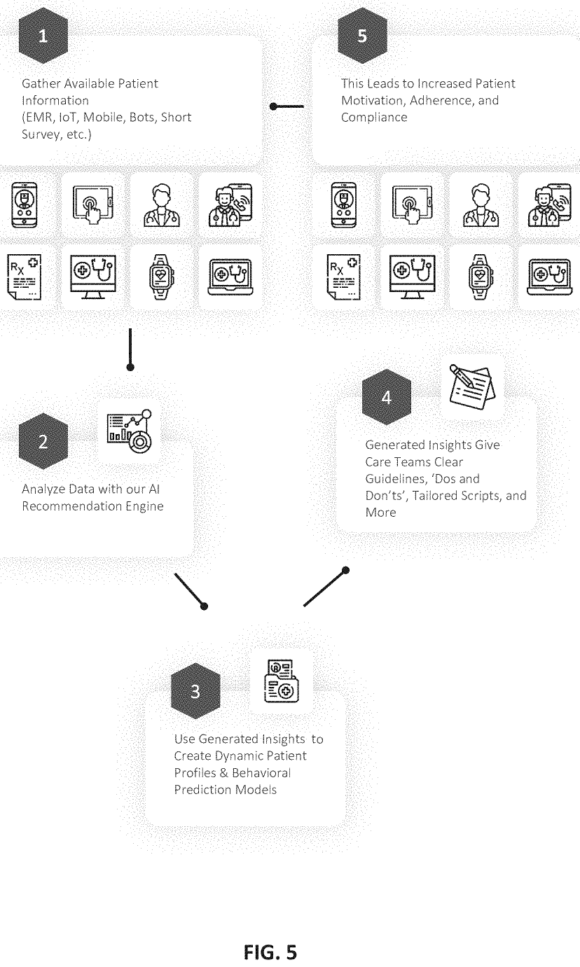

Reference is also made to FIG. 5 which is a basic flow chart of a further additional exemplary process for generating messages for user interaction, according to some embodiments of the present invention.

The flow may start by gathering available information about the subject, or the patient in this example, from sources such as electronic medical records (EMR), medical exams, measurements from hand held, wearable, and other devices, for example using IoT, mobile telephones, web bots, short manual surveys, and/or the like.

The flow may continue by Analyzing data using the disclosed recommendation engine.

The flow may continue by using insights generated by the recommendation engine to create dynamic patient profiles comprising behavioral prediction models, using artificial intelligence, expert knowledge representation, or combination thereof.

The generated insights may be used for automatic messages generation aimed at the subject, family members thereof, and/or give care teams clear guidelines, for example, guidelines colloquially named ‘Dos and Don'ts’, provide tailored scripts for conversations, and/or the like.

Followingly, adherence and other medical measurements may be used as feedback loop to one or more reruns of the further additional exemplary process for generating messages for user interaction.

The disclosed method has been shown to leads to increased patient motivation, adherence, and compliance, for example from 29% to 60% in Priority Life Care, and form 25% to 75% in Sheba Medical Center, with 87% patient engagement and provider satisfaction, and 80% accuracy and patient satisfaction.

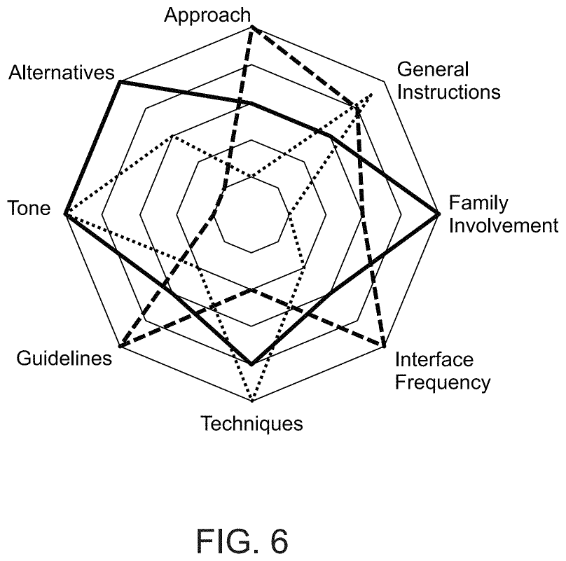

Reference is now made to FIG. 6 which is an exemplary chart of exemplary subject traits, according to some embodiments of the present invention.

The chart shows exemplary traits exemplary traits for three different subject. For example, the first patient, marked by the brighter lines, may have average family involvement, and be characterized by low tone, yet high attention to guidelines.

Such a subject may receive messages such as “You are not alone”, messages comprising detailed guidelines, messages aimed to strengthen self-ability with examples. The subject may benefit from high interface frequency.

Another subject, marked by the moderate lines, may have little or no family involvement, and be attuned to techniques and instructions. This subject may best benefit from emphasis on tools & techniques, presenting goals and discuss end-results.

Yet another subject, marked by the thick dark lines, may be have high family involvement, and interest in alternative methods. The messages apt for this subject, may be characterized by emphasis on alternatives and general instructions. These messages may present mid-goals only, and family involvement may also be used to promote adherence to the care program.

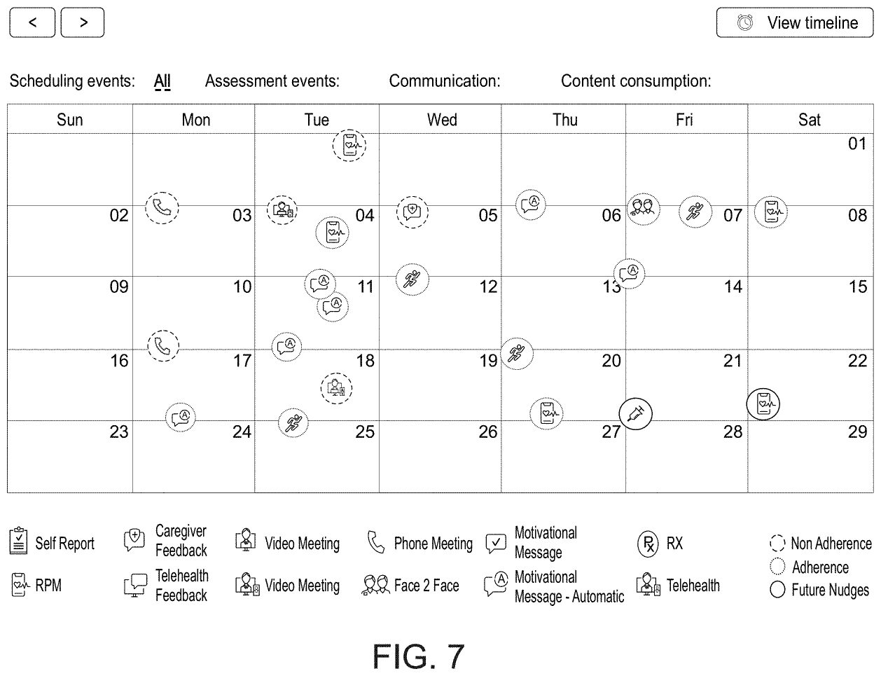

Reference is also made to FIG. 7 which is a basic flow chart of an exemplary month of an exemplary care program, according to some embodiments of the present invention.

In this exemplary monthly schedule, phone meetings with care staff planned for Monday were missed, however a face to face meeting on Friday, for example took place. An RPM measurement scheduled is yet to be done.

A measure of adherence score, measuring one or more aspects of the subject's adherence and persistence to the care program may be calculated and reported all the reported in a frequency adjustable by factors depending on the care program type, subject state, conditions, and/or the like. Exemplary frequencies may be every 3 days, weekly, or monthly. Therefore internally stored details may comprise a weighted analysis of previous subject's adherence to the prescribed treatments, using the data collectors

The measure of adherence score may be evaluated using weighted techniques, wherein care staff, experts, and/or the like may determine the importance level per each adherence factor. For example, a medic may decide that data regarding taking pills is more significant then diet information by ratio of 2:1, and hierarchy of weighted techniques may be supported.

Compared to methods wherein the information may be presented to the therapist, trainer, group conductor, or the like, by a plurality of graphs per topic, which may be time consuming and confusing, the disclosure enables reviewing fewer or a single graph, for getting the overall picture on the subject's adherence to the care program.

On Tuesday, automatic motivational messages were sent. Examples for phrasings of motivational messages may include “It's in your hands, and you have the power. As a doer, define the mission for yourself”, for a more independent subject, “Proud of you for making the decision to take the meds as prescribed. It's proof of strong character. Keep it up!”, and for example, when an indication a weight control goal was not met “You're a responsible person. Let it show in your nutrition too. Don't give up on yourself.”

It is expected that during the life of a patent maturing from this application many relevant multimedia and messaging methods will be developed and the scopes of the terms multimedia and message are intended to include all such new technologies a priori.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of generating motivational interactions for increasing a subject adherence to a prescribed treatments, the method comprising:
extracting a plurality of metrics estimating the subject psychological and motivational traits using data collectors;
using a profiler to extract a subject profile, based on the plurality of metrics and a plurality of internally stored details;
using a collaborative filtering mechanism based recommender to generate a plurality of recommended personalized interventions;
applying a plurality of rules using an expert-in-the-middle on the plurality of recommended personalized interventions; and
generating motivational interactions according to the plurality of recommended personalized interventions.

2. The method of claim 1, wherein the extracting a plurality of metrics comprises analyzing specialized self-questionnaire.

3. The method of claim 1, further comprising data collectors, comprising tracing the subject data from the subject wearable device or mobile phone.

4. The method of claim 3, wherein the internally stored details comprise a weighted analysis of previous subject's adherence to the prescribed treatments, using the data collectors.

5. The method of claim 1, further comprising initializing an inference of effectiveness of the plurality of recommended personalized interventions using expert-in-the-middle.

6. The method of claim 1, wherein the motivational interactions comprise motivational and informational messages and notifications to increasing compliance to a prescribed treatments for the subject.

7. The method of claim 1, further comprising initializing the collaborative filtering mechanism based recommender using the expert-in-the-middle.

8. The method of claim 7, further comprising adjusting the collaborative filtering mechanism based recommender using at least one questionnaire answered by the subject.

9. The method of claim 1, further comprising initializing the collaborative filtering mechanism based recommender using specialized cold start mechanism.

10. The method of claim 1, wherein generating motivational interactions or extracting a plurality of metrics further comprising using a chat-bot configured by the subject profile.

11. A system for generating motivational interactions for increasing a subject adherence to a prescribed treatments, comprising at least one hardware processor configured to:
extracting a plurality of metrics estimating the subject psychological and motivational traits using data collectors;
using a profiler to extract a subject profile, based on the plurality of metrics and a plurality of internally stored details;
using a collaborative filtering mechanism based recommender to generate a plurality of recommended personalized interventions;
applying a plurality of rules using an expert-in-the-middle on the plurality of recommended personalized interventions; and
generating motivational interactions according to the plurality of recommended personalized interventions.

12. The system of claim 11, wherein the extracting a plurality of metrics comprises analyzing specialized self-questionnaire.

13. The system of claim 11, further comprising data collectors, comprising tracing the subject data from the subject wearable device or mobile phone.

14. The system of claim 13, wherein the internally stored details comprise a weighted analysis of previous subject's adherence to the prescribed treatments, using the data collectors.

15. The system of claim 11, further comprising initializing an inference of effectiveness of the plurality of recommended personalized interventions using expert-in-the-middle.

16. The system of claim 11, wherein the motivational interactions comprise motivational and informational messages and notifications to increasing compliance to a prescribed treatments for the subject.

17. The system of claim 11, further comprising initializing the collaborative filtering mechanism based recommender using the expert-in-the-middle.

18. The system of claim 17, further comprising adjusting the collaborative filtering mechanism based recommender using at least one questionnaire answered by the subject.

19. The system of claim 11, further comprising initializing the collaborative filtering mechanism based recommender using specialized cold start mechanism.

20. The system of claim 11, wherein generating motivational interactions or extracting a plurality of metrics further comprising using a chat-bot configured by the subject profile.
